(19) United States
(12) Patent Application Publication
(10) Pub. No.: US 2009/0143496 A1
(43) Pub. Date: Jun. 4, 2009

(54) CROSSLINKABLE SILOXANE-UREA COPOLYMERS

(75) Inventor: Wolfgang Ziche, Burghausen (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 11/719,269

(22) PCT Filed: Nov. 24, 2005
(86) PCT No.: PCT/EP05/12585

§ 371 Date: May 14, 2007

(30) Foreign Application Priority Data
Dec. 2, 2004 (DE) 10 2004 058 193.2

(51) Int. Cl. C08G 18/61; C07C 265/02

(52) U.S. Cl. 522/148; 528/25; 560/355

(57) ABSTRACT

Siloxane/urea copolymers contain pendant unsaturated groups crosslinkable by actinic radiation. The copolymers are suitable for use as non-creeping hotmelt adhesives and for other uses as well.

The invention relates to diisocyanates having radiation-curable groups and to radiation-crosslinkable organopolysiloxane-polyurea block copolymers obtainable therefrom, and to their use.

Organopolysiloxane-polyurea block copolymers are known and can be prepared from aminoalkyl-terminated siloxanes and diisocyanates.

The formation of hydrogen bonds between the urea groups allow such polymers to be thermoplastic elastomers; in other words, they are plastic above the softening point, while below it they have elastic properties. Hence they can be used, for example, as hotmelt adhesives. A disadvantage of this is that the adhesive bond is reversible by an increase in temperature beyond the softening point. Moreover, moldings or adhesive bonds produced from such polymers are subject to cold flow, because even below the softening point hydrogen bonds are able continually to separate and reattach, so that deformation and hence failure of the desired function are possibilities. Accordingly the field of use is limited to applications where no heightened temperatures and/or forces act on the thermoplastic elastomer.

One solution to the problem is to crosslink the individual polymer chains additionally with covalent bonds, i.e., thermally irreversible bonds. If, in production, the thermoplastic elastomers are crosslinked through the use, for example, of trifunctional units, the processing properties (e.g., melt viscosity) are adversely affected. Crosslinking after application is therefore more sensible. Reaction products of polyisocyanates, isocyanurate triisocyanates for example, with hydroxyl-containing acrylates are known (JP 2004137439 A2, JP 2004143303 A2), though only the isocyanate-free urethane acrylates (EP 430209 A2) are prepared and used. Light-curable elastomers are known and are described for example in DE 42 11 391 A1. Photocurable liquid silicone compositions are described for example in DE 697 17 935 T2 and U.S. Pat. No. 5,635,544. Light-sensitive thermoplastic compositions are described in DE 689 06 723 T2.

The invention provides copolymers of the general formula

R′-[(A)ₐ(B)_{b}(C)_{c}]-R″ (II),

in which

(A) can be alike or different and is a unit of the formula (III)

—[CO—NH-Z(L)ᵣ-NH—CO—ND-Y—Si(OR¹)ₒR₂₋ₒ—(O—SiR_{q}R⁵_{2-q})ₙ—O—Si(OR¹)ₒR₂₋ₒ—Y-ND]-,

(B) can be alike or different and is a unit of the formula

—[CO—NH-Z(L)ᵣ-NH—CO—NR⁴-G-NR⁴]— (IV)

and

(C) can be alike or different and is a unit of the formula

—[CO—NH-Z(L)ᵣ-NH—CO-E-X-E]- (V)

where
X can be alike or different and is an alkylene radical which has 1 to 700 carbon atoms, which is unsubstituted or substituted by fluorine, chlorine, C₁-C₆ alkyl or C₁-C₆ alkyl ester and in which methylene units not adjacent to one another can be replaced by groups —O—, —COO—, —OCO— or —OCOO—, or is unsubstituted or substituted arylene radical having 6 to 22 carbon atoms,
Y can be alike or different and is a divalent hydrocarbon radical which has 1 to 30 carbon atoms and in which methylene units not adjacent to one another can be replaced by groups —O—, or is the radical —(CH₂)₃—NH—SiR₂—(CH₂)₃—NH—,
Z can be alike or different and is an (r+2)-valent, unsubstituted or substituted hydrocarbon radical which has 1 to 60 carbon atoms and may be interrupted by heteroatoms,
L can be alike or different and is a radical —NH—(C═O)-E-Q-E-(C═O)—CR⁶═CR⁷₂,
Q is a divalent, unsubstituted or substituted hydrocarbon radical having 1 to 30 carbon atoms and may be interrupted by heteroatoms,
R⁶ is hydrogen atom or a monovalent hydrocarbon radical which is unsubstituted or substituted by fluorine or chlorine and has 1 to 20 carbon atoms,
R⁷ can be alike or different and is hydrogen atom or a monovalent hydrocarbon radical which is unsubstituted or substituted by fluorine or chlorine and has 1 to 20 carbon atoms,
r can be alike or different and is 0 or an integer of at least 1,
D can be alike or different and is hydrogen atom or a monovalent, unsubstituted or substituted hydrocarbon radical,
E can be alike or different and is an oxygen atom or an amino group -ND-,
R can be alike or different and is a monovalent hydrocarbon radical which has 1 to 20 carbon atoms and is unsubstituted or substituted by fluorine or chlorine,
R⁵ can be alike or different and is a monovalent, C═C-unsaturated hydrocarbon radical which has 2 to 20 carbon atoms, is unsubstituted or substituted by fluorine, chlorine or oxygen, and is uninterrupted or interrupted by oxygen,

q is 0, 1 or 2

R¹ can be alike or different and is hydrogen atom or a monovalent hydrocarbon radical which has 1 to 20 carbon atoms and is unsubstituted or substituted by fluorine, chlorine or organyloxy groups, or is —(C═O)—R or —N═CR₂,
R⁴ can be alike or different and is a radical of the formula -Z′-SiRₚ(OR¹)₃₋ₚ with Z′ equal to the definition specified below, and p being 0, 1 or 2, or is hydrogen atom or a monovalent, unsubstituted or substituted hydrocarbon radical,
Z′ can be alike or different and is divalent, unsubstituted or substituted hydrocarbon radical having 1 to 30 carbon atoms,
G can be alike or different and is a divalent, unsubstituted or substituted hydrocarbon radical having 1 to 60 carbon atoms and may be interrupted by heteroatoms,
R″ is hydrogen atom or a radical —CO—NH-Z(L)ᵣ-NCO, preferably hydrogen atom,
R′ if R″ is hydrogen atom is a radical HND-Y—Si(OR¹)ₒR₂₋ₒ—(O—SiR₂)ₙ—O—Si(OR¹)ₒR₂₋ₒ—Y-ND-, HNR⁴-G-NR⁴— or HE-X-E-, preferably HND-Y—Si(OR¹)ₒR₂₋ₒ —(O—SiR₂)ₙ—O—Si(OR¹)ₒR₂₋ₒ—Y-ND- or HNR⁴-G-NR⁴, and
   if R″ is radical —CO—NH-Z(L)ᵣNCO has the definition of radical OCN-Z(L)-NH—CO-ND-Y—Si(OR¹)ₒR₂₋ₒ—(O—SiR₂)ₙ—O—Si(OR¹)ₒR₂₋ₒ—Y-ND-, OCN-Z(L)ᵣ-NH—CO—NR⁴-G-NR⁴— or OCN-Z(L)ᵣ-NH—CO-E-X-E-, preferably OCN-Z(L)ᵣ-NH—CO-ND-Y—Si(OR)ₒR₂₋ₒ—(O—SiR₂)ₙ—O—Si(OR¹)ₒR₂₋ₒ—Y-ND- or OCN-Z(L)ᵣ-NH—CO—NR⁴-G-NR⁴—,
n can be alike or different and is an integer from 1 to 4000,
o can be alike or different and is 0, 1 or 2, preferably 0,
a is an integer of at least 1,
b is 0 or an integer of at least 1,
c is 0 or an integer of at least 1,
   with the proviso that in the molecule there is at least one radical L and also the individual blocks (A), (B) and (C) can be distributed randomly in the polymer.

For the purposes of the present invention the term “organopolysiloxanes” is intended to encomPass polymeric, oligomeric, and dimeric siloxanes.

Examples of (2+r)-valent radicals Z are alkylene radicals, such as the methylene, ethylene, n-propylene, isopropylene, n-butylene, isobutylene, tert-butylene, n-pentylene, isopentylene, neopentylene, tert-pentylene radical, hexylene radicals, such as the n-hexylene radical, heptylene radicals, such as the n-heptylene radical, octylene radicals, such as the n-octylene radical and isootcylene radicals, such as the 2,2,4-trimethylpentylene radical, nonylene radicals, such as the n-nonylene radical, decylene radicals, such as the n-decylene radical, dodecylene radicals, such as the n-dodecylene radical; alkenylene radicals, such as the vinylene and the allylene radical; cycloalkylene radicals, such as cyclopentylene, cyclohexylene, cycloheptylene radicals and methylcyclohexylene radicals; arylene radicals, such as the phenylene and the naphthylene radical; alkarylene radicals, such as o-, m-, p-tolylene radicals, xylylene radicals and ethylphenylene radicals; aralkylene radicals, such as the benzylene radical, the α- and the β-phenylethylene radical, and also the 4,4′-methylenediphenylene radical; trivalent 4,4′,4″-triphenylmethane radical, trivalent isocyanurate radicals which can be prepared by reacting di- or polyisocyanates with themselves in the presence of a trimerization catalyst. Isocyanates containing such isocyanurate radicals are available commercially, as for example the isocyanurates of isophorone diisocyanate having a nominal functionality of 3.0 (available commercially under the name “Vestanat T1890” from Degussa AG, Germany) or of hexamethylene diisocyanate having a nominal functionality of 3.0 (available commercially under the name “Desmodur N3600” from Bayer AG, Germany).

Radical Z preferably comprises alkylene groups having 1 to 24 carbon atoms and trivalent trialkylene-isocyanurate radicals, more preferably hexylene, 4,4′-methylenebiscyclohexylene, 3-methylene-3,5,5-tri-methylcyclohexylene radical, and the trivalent trishexylisocyanurate radical.

Examples of the divalent radicals Q are the divalent groups specified for Z.

Radical Q preferably comprises unsubstituted or substituted alkylene radicals having 1 to 30 carbon atoms, which may be interrupted by heteroatoms, or comprises arylene radicals having 6 to 22 carbon atoms. With particular preference radical Q comprises alkylene groups having 1 to 12 carbon atoms, especially alkylene groups having 1 or 4 carbon atoms.

Examples of the divalent radicals G are the divalent examples listed for Z. Radical G preferably comprises alkylene radicals having 1 to 6 carbon atoms, arylene radicals such as the o-, m-, or p-phenylene radical, and aralkylene radicals such as the phenylethylene radical, with radical —CH₂CH₂— being particularly preferred.

Examples of Z′ are all divalent examples stated for Z. Radical Z′ preferably comprises alkylene groups having 1 to 24 carbon atoms, more preferably alkylene groups having 1 or 3 carbon atoms.

Examples of Y are all divalent examples specified for Z. Radical Y preferably comprises alkylene radicals having 1 to 30 carbon atoms, in which methylene units not adjacent to one another can be replaced by groups —O—, or comprises arylene radicals having 6 to 22 carbon atoms. With particular preference radical Y comprises alkylene groups having 1 to 6 carbon atoms, especially alkylene groups having 1 or 3 carbon atoms.

Examples of radical X are the butylene radical, ethylene radical, hexylene radical, and radicals of the formulae —(CH₂)₃—(O—CH(CH₃)—CH₂)₂₋₃₀₀₀—O—(CH₂)₃—, —CH(CH₃)—CH₂—(O—CH(CH₃)—CH₂)₂₋₃₀₀₀—, —(CH₂)₃—(O—CH₂—CH₂)₂₋₃₀₀—O—(CH₂)₃—, and —CH₂—CH₂—(OCH₂—CH₂)₂₋₃₀₀—.

Radical X preferably comprises polyether radicals, more preferably polypropylene glycol radicals, especially those having 2 to 600 carbon atoms.

Examples of radical R are alkyl radicals, such as the methyl, ethyl, n-propyl, isopropyl, n-butyl, isobutyl, tert-butyl, n-pentyl, isopentyl, neopentyl, tert-pentyl radical, hexyl radicals, such as the n-hexyl radical, heptyl radicals, such as the n-heptyl radical, octyl radicals, such as the n-octyl radical and isooctyl radicals, such as the 2,2,4-trimethylpentyl radical, nonyl radicals, such as the n-nonyl radical, decyl radicals, such as the n-decyl radical, dodecyl radicals, such as the n-dodecyl radical; alkenyl radicals, such as the vinyl and the allyl radical; cycloalkyl radicals, such as cyclopentyl, cyclohexyl, cycloheptyl radicals and methylcyclohexyl radicals; aryl radicals, such as the phenyl and the naphthyl radical; alkaryl radicals, such as o-, m-, p-tolyl radicals, xylyl radicals and ethylphenyl radicals; aralkyl radicals, such as the benzyl radical, the α- and the β-phenylethyl radical.

With preference radical R is a hydrocarbon radical having 1 to 6 carbon atoms, more preferably an alkyl radical having 1 to 4 carbon atoms, especially the methyl radical.

Examples of radical R⁵ are vinyl, allyl, 1,3-butadienyl, cyclohexenyl, 4-styryl, acryloyloxy-methyl, acryloyloxypropyl, methacryloyloxymethyl and methacryloyloxypropyl radical, where vinyl and allyl radical are preferred and vinyl radical is particularly preferred.

Examples of radical R¹ are the examples specified for radical R, and also alkoxyalkyl radicals.

With preference radical R¹ comprises linear or branched alkyl radicals having 1 to 12 carbon atoms and alkoxyalkyl radicals such as the 2-methoxyethyl, 2-ethoxyethyl, and 2-(2′-methoxyethyl)ethyl radical, more preferably alkyl radicals having 1 to 12 carbon atoms, especially the methyl and ethyl radical.

Examples of radical R⁴ are the radicals specified for R, hydrogen atom, and also the radicals —(CH₂)₄Si(OCH₃)₃, —(CH₂CH(CH₃)CH₂)Si(OCH₃)₃, —(CH₂CH(CH₃)CH₂)Si(OCH₂CH₃)₃, —(CH₂CH(CH₃)CH₂)SiCH₃(OCH₃)₂, —(CH₂CH(CH₃)CH₂)SiCH₃(OCH₂CH₃)₃, —(CH₂)₃Si(OCH₃)₃, —(CH₂)₃Si(OCH₂CH₃)₃, —(CH₂)₃SiCH₃(OCH₃)₂, —(CH₂)₃SiCH₃(OCH₂CH₃)₂, —CH₂Si(OCH₃)₃, —CH₂Si(OCH₂CH₃)₃, —CH₂SiCH₃(OCH₃)₂, —CH₂SiCH₃ (OCH₃)₂, —(CH₂)₃SiCH₃(OCH₂CH₂OCH₃)₂, and —C₆H₄—(CH₂)₂SiCH₃(OCH₂CH₃)₂.

Preferably radical R⁴ comprises hydrogen atom and the above-indicated silyl-substituted alkyl radicals, more preferably hydrogen atom and the radicals —(CH₂)₃Si(OCH₃)₃, —(CH₂)₃Si(OCH₂CH₃)₃, —(CH₂)₃SiCH₃(OCH₃)₂, —(CH₂)₃SiCH₃(OCH₂CH₃)₂, —CH₂Si(OCH₃)₃, —CH₂Si(OCH₂CH₃)₃, —CH₂SiCH₃(OCH₃)₂, and —CH₂SiCH₃ (OCH₃)₂.

Examples of radical R⁶ are the radicals specified for R, plus hydrogen atom. Preferably radical R⁶ is hydrogen atom and methyl radical.

Examples of radical R⁷ are the radicals specified for R, plus hydrogen atom. Preferably radical R⁷ is hydrogen atom.

Examples of hydrocarbon radicals D are the radicals specified above for R.

Radical D is preferably an alkyl radical or hydrogen atom, more preferably an alkyl radical having 1 to 12 carbon atoms or hydrogen atom, in particular hydrogen atom.

Radical E is preferably oxygen atom.

Radical L is preferably —NH—(C═O)—O—CH₂—CH₂—O—(C═O)—CH═CH₂ or —NH—(C═O)—O—CH₂—CH₂—O—(C═O)—C(CH₃)═CH₂.

Preferably a has the definition of an integer from 1 to 1000, more preferably from 5 to 1000, especially 5 to 100.

Preferably b has the definition of 0 or an integer from 1 to 1000, more preferably 0 or an integer from 5 to 1000, especially 0.

Preferably c has the definition of 0 or an integer from 1 to 100, more preferably 0 or an integer from 1 to 10, especially 0.

Preferably r has the definition of an integer from 1 to 100, more preferably from 1 to 10, especially 1.

Examples of radicals R′ are, if R″ is hydrogen atom, radicals which result from the unreacted end groups of the reactants employed, such as H₂N—(CH₂)₃—Si(CH₃)₂—(O—Si(CH₃)₂)₄₀—O—Si(CH₃)₂—(CH₂)₃—NH—, H₂N—CH₂—CH₂—N—(CH₂)₃Si(OCH₃)₃, and HO—(CH₂CH(CH₃)O)₅₀—.

Examples of radicals R′ are, if R″ is —CO—NH-Z(L)ᵣ-NCO, radicals which result from the unreacted end groups of the reactants employed, such as

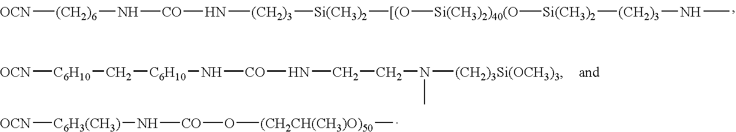

Examples of radicals R″ are hydrogen atom, —CO—NH—(CH₂)₆—NCO, —CO—NH—C₆H₁₀—CH₂—C₆H₁₀—NCO and —CO—NH—C₆H₃(CH₃)—NCO.

Preferably n is an integer from 10 to 4000, more preferably from 30 to 1000.

Index p is preferably 0.

Copolymers of the invention comprising units (C) may give rise to a harder material in comparison to copolymers of the invention that comprise no unit (C), since there are more hydrogen bonds in them. If the proportion of component (C) becomes too high, separation phenomena occur between the organic and polysiloxane constituents, so that the transparency of the copolymers of the invention is reduced and the copolymers become turbid. Preference is given to polymers of the formula (II) with c as zero, since consequently there are exclusively siloxane chains present and the polymers as a result have advantages, such as high transparency and UV-stability in conjunction with low surface energies, for example.

Examples of the inventive copolymers of the formula (II) are H[NH(CH₂)₃SiMe₂O(SiMe₂O)₃₅SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O(SiMe₂O)₃₅SiMe₂(CH₂)₃—NH₂,

H[NH(CH₂)₃Si(OMe)₂O(SiMe₂O)₃₅Si(OMe)₂(CH₂)₃—NH—CO—NH—(CH₂)—₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O—(SiMe₂O)₃₅SiMe₂(CH₂)₃—NH₂,

H[[NH(CH₂)₃SiMe₂O(SiMe₂O)₃₅SiMe₂(CH₂)₃—NH—CO—NH—(CH₂)—₆-cyclo{N(C═O)—N—[H₂C═C(CH₃)C(═O)—O(CH₂)₂N(C(CH₃)₃) C(═O)NH—(CH₂)₆](C═O)—N(C═O)}—(CH₂)₆—NH—CO—]₅—[NH—CH₂CH₂—NH—CO—NH—(CH₂)—₆-cyclo{N(C═O)—N—[H₂C═C(CH₃)C(═O)—O(CH₂)₂N(C(CH₃)₃)C(═O)NH—(CH₂)₆](C═O)—N(C═O)}-(CH₂)₆—NH—CO]₅]₁₀—NH—CH₂CH₂—NH₂,

H [NH(CH₂)₃SiMe₂O(SiMe₂O)₃₅SiMe₂(CH₂)₃—NH—CO—NH—(CH₂) 6-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—O—(CH₂CH₂O)₅—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O(SiMe₂O)₃₅SiMe₂(CH₂)₃—NH₂,

H[NH—CH₂—SiMe₂O(SiMe₂O)₃₅SiMe₂-CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆—NH—CO—NH—(CH₂CH₂O)₅—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}-(CH₂)₆—NH—CO]₁₀—CH₂—SiMe₂O(SiMe₂O)₃₅SiMe₂-CH₂—NH₂,

H[NH(CH₂)₃SiMe₂O(SiMe₂O)₃₅SiMe₂(CH₂)₃—NH—CO—NH—(CH₂)—₆-cyclo{N(C═O)—N—[H₂C═C(CH₃)C(═O)—O(CH₂)₂OC(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂C(═O)NH—(CH₂)₆](C═O)—N(C═O)}—(CH₂)₆—NH—CO]₁₀—NH(CH₂)₃SiMe₂O(SiMe₂O)₃₅SiMe₂(CH₂)₃—NH₂,

OCN—C₇H₆—NH—CO—[NH(CH₂)₃SiMe₂O(SiMe₂O)₃₅SiMe₂(CH₂)₃—NH—CO—NH—(CH₂)—₆-cyclo{N(C═O)—N—[H₂C═CH—C(═O)—O(CH₂)₂OC(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO]₁₀—NH(CH₂)₃SiMe₂O(SiMe₂O)₃₅SiMe₂(CH₂)₃—NH—CO—NH—(CH₂)—₆-cyclo{N(C═O)—N—[H₂C═CH—C(═O)—O(CH₂)₂C(═O)NH—(CH₂)₆](C═O)—N(C═O)}—(CH₂)₆—NCO, and

OCN—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO—[NH(CH₂)₃SiMe₂O(SiMe₂O)₃₅SiMe₂(CH₂)₃—NH—CO—NH—(CH₂)—₆-cyclo{N(C═O)—N—[H₂C═CH—C(═O)—O(CH₂)₂C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO]₁₀—NH—CH₂CH₂—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NCO,

H[NH(CH₂)₃SiMe₂O(SiMe₂O)₃₅SiMe₂(CH₂)₃—NH—CO—NH—(C₆H₄)CH—[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₅SiMe₂(CH₂)₃—NH₂,

H[NH(CH₂)₃Si(OMe)₂O(SiMe₂O)₃₅Si(OMe)₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₅SiMe₂(CH₂)₃—NH₂,

H[[NH(CH₂)₃SiMe₂O(SiMe₂O)₃₅SiMe₂(CH₂)₃—NH—CO—NH—(C₆H₄)CH—[H₂C═C(CH₃)C(═O)—O(CH₂)₂N(C(CH₃)₃)C(═O)NH—(C₆H₄)](C₆H₄)—NH—CO—]₅—[NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₅]₁₀—NH—CH₂CH₂—NH₂,

H[NH(CH₂)₃SiMe₂O(SiMe₂O)₃₅SiMe₂(CH₂)₃—NH—CO—NH—(C₆H₄)CH—[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—O—(CH₂CH₂O)₅—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₅SiMe₂(CH₂)₃—NH₂,

H[NH—CH₂—SiMe₂O(SiMe₂O)₃₅SiMe₂-CH₂—NH—CO—NH—(C₆H₄)CH—[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—(CH₂CH₂O)₅—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—CH₂—SiMe₂O(SiMe₂O)₃₅SiMe₂-CH₂—NH₂,

H[NH(CH₂)₃SiMe₂O(SiMe₂O)₃₅SiMe₂(CH₂)₃—NH—CO—NH—(C₆H₄)CH—[(H₂C═C(CH₃)C(═O)—O(CH₂)₂OC(═O)NH—C₆H₄)](C₆H₄)—NH—CO—NH—CH₂CH₂—NH—CO—NH—(C₆H₄)CH[(H₂C═C(CH₃)C(═O)—O(CH₂)₂OC(═O)NH—C₆H₄)](C₆H₄)—NH—CO]₁₀—NH(CH₂)₃SiMe₂O(SiMe₂O)₃₅SiMe₂(CH₂)₃—NH₂,

OCN—(C₆H₄)CH[(H₂C═CH—C(═O)—O(CH₂)₂OC(═O)NH—C₆H₄)](C₆H₄)—NH—CO—[NH(CH₂)₃SiMe₂O(SiMe₂O)₃₅SiMe₂(CH₂)₃—NH—CO—NH—(C₆H₄)CH [(H₂C═CH—C(═O)—O(CH₂)₂OC(═O)NH—C₆H₄)](C₆H₄)—NH—CO—NH—CH₂CH₂—NH—CO—NH—(C₆H₄)CH[(H₂C═CH—C(═O)—O(CH₂)₂OC(═O)NH—C₆H₄)](C₆H₄)—NH—CO]₁₀—NH(CH₂)₃SiMe₂O(SiMe₂O)₃₅SiMe₂(CH₂)₃—NH—CO—NH—(C₆H₄)CH[(H₂C═CH—C(═O)—O(CH₂)₂OC(═O)NH—C₆H₄)](C₆H₄)—NCO, and

OCN—(C₆H₄)CH[(H₂C═CH—C(═O)—O(CH₂)₂OC(═O)NH—C₆H₄)](C₆H₄)—NH—CO—[NH(CH₂)₃SiMe₂O(SiMe₂O)₃₅SiMe₂(CH₂)₃—NH—CO—NH—(C₆H₄)CH[(H₂C═CH—C(═O)—O(CH₂)₂OC(═O)NH—C₆H₄)](C₆H₄)—NH—CO—NH—CH₂CH₂—NH—CO—NH—(C₆H₄)CH[(H₂C═CH—C(═O)—O(CH₂)₂OC(═O)NH—C₆H₄)](C₆H₄)—NH—CO]₁₀—NH—CH₂CH₂—NH—CO—NH—(C₆H₄)CH[(H₂C═CH—C(═O)—O(CH₂)₂OC(═O)NH—C₆H₄)](C₆H₄)—NCO.

H[NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O(SiMe₂O)₃₃-(SiMeViO)₂SiMe₂(CH₂)₃—NH₂,

H[NH(CH₂)₃Si(OMe)₂O(SiMe₂O)₃₃(SiMeViO)₂Si(OMe)₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O(SiMe₂O)₃₃-(SiMeViO)₂SiMe₂(CH₂)₃—NH₂,

H[[NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—]₅—[NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₅]₁₀—NH—CH₂CH₂—NH₂,

H[NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—O—(CH₂CH₂O)₅—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O(SiMe₂O)₃₃(SiMeViO)₂SiMe₂(CH₂)₃—NH₂,

H[NH—CH₂—SiMe₂O(SiMe₂O)₃₃ (SiMeViO)₂SiMe₂-CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆—NH—CO—NH—(CH₂CH₂O)₅—CH₂CH₂—NH—CO—NH—(CH₂)—₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—CH₂—SiMe₂O(SiMe₂O)₃₃(SiMeViO)₂SiMe₂-CH₂—NH₂,

H[NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═C(CH₃)C(═O)—O(CH₂)₂C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂OC(═O)NH—(CH₂)₆](C═O)—N(C═O)}—(CH₂)₆—NH—CO]₁₀—NH(CH₂)₃SiMe₂O(SiMe₂O)₃₃(SiMeViO)₂SiMe₂(CH₂)₃—NH₂,

OCN—C₇H₆—NH—CO—[NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NH—CO]₁₀—NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NCO, and

OCN—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO—[NH(CH₂)₃SiMe₂O(SiMe₂O)₃₃(SiMeViO)₂SiMe₂(CH₂)₃—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO]₁₀—NH—CH₂CH₂—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NCO,

H[NH(CH₂)₃SiMe₂O(SiMe₂O)₃₃(SiMeViO)₂SiMe₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₃(SiMeViO)₂SiMe₂(CH₂)₃—NH₂,

H[NH(CH₂)₃Si(OMe)₂O(SiMe₂O)₃₃(SiMeViO)₂Si(OMe) 2(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₃(SiMeViO)₂—SiMe₂(CH₂)₃—NH₂,

H[[NH(CH₂)₃SiMe₂O(SiMe₂O)₃₃(SiMeViO)₂SiMe₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—]₅—[NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₅]₁₀—NH—CH₂CH₂—NH₂,

H[NH(CH₂)₃SiMe₂O(SiMe₂O)₃₃(SiMeViO)₂SiMe₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—O—(CH₂CH₂O)₅—CO—NH—(C₆H₄)CH—[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₃(SiMeViO)₂SiMe₂(CH₂)₃—NH₂,

H[NH—CH₂—SiMe₂O(SiMe₂O)₃₃(SiMeViO)₂SiMe₂-CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—(CH₂CH₂O)₅—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—CH₂—SiMe₂O(SiMe₂O)₃₃(SiMeViO)₂SiMe₂-CH₂—NH₂,

H[NH(CH₂)₃SiMe₂O(SiMe₂O)₃₃ (SiMeViO)₂SiMe₂(CH₂)₃—NH—CO—NH—(C₆H₄)CH[(H₂C═C(CH₃)C(═O)—O(CH₂)₂OC(═O)NH—C₆H₄)](C₆H₄)—NH—CO—NH—CH₂CH₂—NH—CO—NH—(C₆H₄)CH[(H₂C═C(CH₃)C(═O)—O(CH₂)₂OC(═O)NH—C₆H₄)](C₆H₄)—NH—CO]₁₀—NH(CH₂)₃SiMe₂O(SiMe₂O)₃₃(SiMeViO)₂SiMe₂(CH₂)₃—NH₂,

OCN—(C₆H₄)CH[(H₂C═CH—C(═O)—O(CH₂)₂OC(═O)NH—C₆H₄)](C₆H₄)—NH—CO—[NH(CH₂)₃SiMe₂O(SiMe₂O)₃₃ (SiMeViO)₂SiMe₂(CH₂)₃—NH—CO—NH—(C₆H₄)CH[(H₂C═CH—C(═O)—O(CH₂)₂OC(═O)NH—C₆H₄)](C₆H₄)—NH—CO—NH—CH₂CH₂—NH—CO—NH—(C₆H₄)CH[(H₂C═CH—C(═O)—O(CH₂)₂C(═O)NH—C₆H₄)](C₆H₄)—NH—CO]₁₀—NH(CH₂)₃SiMe₂O(SiMe₂O)₃₃(SiMeViO)₂SiMe₂(CH₂)₃—NH—CO—NH—(C₆H₄)CH[(H₂C═CH—C(═O)—O(CH₂)₂OC(═O)NH—C₆H₄)](C₆H₄)—NCO, and

OCN—(C₆H₄)CH[(H₂C═CH—C(═O)—O(CH₂)₂OC(═O)NH—C₆H₄)](C₆H₄)—NH—CO—[NH(CH₂)₃SiMe₂O(SiMe₂O)₃₃(SiMeViO)₂SiMe₂(CH₂)₃—NH—CO—NH—(C₆H₄)CH[(H₂C═CH—C(═O)—O(CH₂)₂C(═O)NH—C₆H₄)](C₆H₄)—NH—CO—NH—CH₂CH₂—NH—CO—NH—(C₆H₄)CH[(H₂C═CH—C(═O)—O(CH₂)₂OC(═O)NH—C₆H₄)](C₆H₄)—NH—CO]₁₀—NH—CH₂CH₂—NH—CO—NH—(C₆H₄)CH[(H₂C═CH—C(═O)—O(CH₂)₂OC(═O)NH—C₆H₄)](C₆H₄)—NCO,

H[NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O) N(C═O)}-(CH₂)₆—NH—CO]₁₀—NH(CH₂)₃SiMe₂O(SiMe₂O)₃₃(SiMeAllO)₂SiMe₂(CH₂)₃—NH₂,

H[NH(CH₂)₃Si(OMe)₂O(SiMe₂O)₃₃(SiMeAllO)₂Si(OMe)₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O(SiMe₂O)₃₃(SiMeAllO)₂SiMe₂(CH₂)₃—NH₂,

H[[NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—]₅—[NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)](C═O)—N(C═O)}—(CH₂)₆—NH—CO]₅]₁₀—NH—CH₂CH₂—NH₂,

H[NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—O—(CH₂CH₂O)₅—CO—NH—(CH₂)—₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—NH(CH₂)₃SiMe₂O-(SiMe₂O)₃₃(SiMeAllO)₂SiMe₂(CH₂)₃—NH₂,

H[NH—CH₂—SiMe₂O(SiMe₂O)₃₃(SiMeAllO)₂SiMe₂-CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO—NH—CH₂CH₂—NH—CO—NH—(CH₂)₆—NH—CO—NH—(CH₂CH₂O)₅—CH₂CH₂—NH—CO—NH—(CH₂)₆-cyclo{N(C═O)—N—[H₂C═C(CH₃)C(═O)—O(CH₂)₂N(C(CH₃)₃)C(═O)NH—(CH₂)₆](C═O)—N(C═O)}—(CH₂)₆—NH—CO]₁₀—CH₂—SiMe₂O(SiMe₂O)₃₃(SiMeAllO)₂SiMe₂-CH₂—NH₂,

H[NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═C(CH₃)C(═O)—O(CH₂)₂OC(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═C(CH₃)C(═O)—O(CH₂)₂C(═O)NH—(CH₂)₆](C═O)—N(C═O)}—(CH₂)₆—NH—CO]₁₀—NH(CH₂)₃SiMe₂O(SiMe₂O)₃₃(SiMeAllO)₂SiMe₂(CH₂)₃—NH₂,

OCN—C₇H₆—NH—CO—[NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NH—CO]₁₀—NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═CH—C(═O)—O(CH₂)₂C(═O)NH—(CH₂)₆](C═O)—N(C═O)}—(CH₂)₆—NCO, and

OCN—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO—[NH(CH₂)₃SiMe₂O(SiMe₂O)₃₃(SiMeAllO)₂SiMe₂(CH₂)₃—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO—NH—CH₂CH₂—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NH—CO]₁₀—NH—CH₂CH₂—NH—CO—NH—(CH₂)₆-cyclo{N(C═O)—N—[H₂C═CH—C(═O)—O(CH₂)₂OC(═O)NH—(CH₂)₆](C═O)—N(C═O)}—(CH₂)₆—NCO,

H[NH(CH₂)₃SiMe₂O(SiMe₂O)₃₃(SiMeAllO)₂SiMe₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₃(SiMeAllO)₂—SiMe₂(CH₂)₃—NH₂,

H[NH(CH₂)₃Si(OMe)₂O(SiMe₂O)₃₃(SiMeAllO)₂Si(OMe)₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₃(SiMeAllO)₂—SiMe₂(CH₂)₃—NH₂,

H[[NH(CH₂)₃SiMe₂O(SiMe₂O)₃₃(SiMeAllO)₂SiMe₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—]₅—[NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO]₅]₁₀—NH—CH₂CH₂—NH₂,

H[NH(CH₂)₃SiMe₂O(SiMe₂O)₃₃(SiMeAllO)₂SiMe₂(CH₂)₃—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—O—(CH₂CH₂O)₅—CO—NH—(C₆H₄)CH[H₂C═C(CH₃)—C(═O)—O(CH₂)₂N(C(CH₃)₃)C(═O)NH—(C₆H₄)](C₆H₄)—NH—CO]₁₀—NH(CH₂)₃SiMe₂O(SiMe₂O)₃₃(SiMeAllO)₂SiMe₂(CH₂)₃—NH₂,

H[NH—CH₂—SiMe₂O(SiMe₂O)₃₃(SiMeAllO)₂SiMe₂-CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—CH₂CH₂—NH—CO—NH—(C₆H₄)CH[H₂C═C(CH₃)C(═O)—O(CH₂)₂N(C(CH₃)₃)C(═O)NH—(C₆H₄)](C₆H₄)—NH—CO—NH—(CH₂CH₂O)₅—CH₂CH₂—NH—CO—NH—(C₆H₄)—CH[H₂C═C(CH₃)C(═O)—O(CH₂)₂N(C(CH₃)₃)C(═O)NH—(C₆H₄)](C₆H₄) NH—CO]₁₀—CH₂—SiMe₂O(SiMe₂O)₃₃(SiMeAllO)₂SiMe₂-CH₂—NH₂,

H[NH(CH₂)₃SiMe₂O(SiMe₂O)₃₃(SiMeAllO)₂SiMe₂(CH₂)₃—NH—CO—NH—(C₆H₄)CH[(H₂C═C(CH₃)C(═O)—O(CH₂)₂OC(═O)NH—C₆H₄)](C₆H₄)—NH—CO—NH—CH₂CH₂—NH—CO—NH—(C₆H₄)CH[(H₂C═C(CH₃)C(═O)—O(CH₂)₂OC(═O)NH—C₆H₄)](C₆H₄)—NH—CO]₁₀—NH(CH₂)₃SiMe₂O(SiMe₂O)₃₃(SiMeAllO)₂SiMe₂(CH₂)₃—NH₂,

OCN—(C₆H₄)CH[(H₂C═CH—C(═O)—O(CH₂)₂OC(═O)NH—C₆H₄)](C₆H₄)—NH—CO—[NH(CH₂)₃SiMe₂O(SiMe₂O)₃₃(SiMeAllO)₂SiMe₂(CH₂)₃—NH—CO—NH—(C₆H₄)CH[(H₂C═CH—C(═O)—O(CH₂)₂OC(═O)NH—C₆H₄)](C₆H₄)—NH—CO—NH—CH₂CH₂—NH—CO—NH—(C₆H₄)CH[(H₂C═CH—C(═O)—O(CH₂)₂OC(═O)NH—C₆H₄)](C₆H₄)—NH—CO]₁₀—NH(CH₂)₃SiMe₂O(SiMe₂O)₃₃(SiMeAllO)₂SiMe₂(CH₂)₃—NH—CO—NH—(C₆H₄)CH[(H₂C═CH—C(═O)—O(CH₂)₂OC(═O)NH—C₆H₄)](C₆H₄)—NCO, and

OCN—(C₆H₄)CH[(H₂C═CH—C(═O)—O(CH₂)₂OC(═O)NH—C₆H₄)](C₆H₄)—NH—CO—[NH(CH₂)₃SiMe₂O(SiMe₂O)₃₃(SiMeAllO)₂SiMe₂(CH₂)₃—NH—CO—NH—(C₆H₄)CH[(H₂C═CH—C(═O)—O(CH₂)₂C(═O)NH—C₆H₄)](C₆H₄)—NH—CO—NH—CH₂CH₂—NH—CO—NH—(C₆H₄)CH[(H₂C═CH—C(═O)—O(CH₂)₂OC(═O)NH—C₆H₄)](C₆H₄)—NH—CO]₁₀—NH—CH₂CH₂—NH—CO—NH—(C₆H₄)CH[(H₂C═CH—C(═O)—O(CH₂)₂OC(═O)NH—C₆H₄)](C₆H₄)—NCO, where Vi is vinyl radical and All is radical —CH₂—CH═CH₂.

The inventive copolymers of the formula (II) contain preferably greater than 70% by weight, more preferably greater than 80% by weight, of units A of the formula (III), based in each case on the total weight of the copolymer.

At room temperature the inventive copolymers of the formula (II) are preferably rubber-elastic solids having tensile strengths of between preferably about 0.5 and 20 MPa and also breaking extensions between preferably about 50% to 1000%. They soften at temperatures between preferably 60 and 200° C., and in so doing they lose their rubber-elastic properties.

By exposure to actinic radiation it is possible for the free-radically crosslinkable radicals L, if desired also the radicals R⁵, to polymerize with one another or with any further crosslinkable C═C groups.

In addition, if hydrolyzable radicals OR¹ are present, they can react to form OH groups as a result of exposure to moisture, which OH groups in turn can condense with further OR¹ or OH groups to form siloxane bonds.

As against the starting polymer prior to crosslinking, these inventive polymers of the formula (II) additionally crosslinked in this way preferably have a softening point which is shifted markedly in the direction of higher temperatures. This opens up the possibility of preparing polymers which can be processed at relatively low temperatures but then in use can be exposed to higher temperatures.

The copolymers of the invention have the advantage, moreover, that they have very good mechanical properties without any need to add fillers.

Furthermore, the copolymers of the invention are distinguished by outstanding physical properties of the kind known for polyorganosiloxanes, such as low glass transition temperatures, transparency, low surface energies, low hydrophobicity, good dielectric properties, and high gas permeability, for example.

Further advantages of the copolymers of the invention are the high thermal and oxidative stability, good stabilities toward swelling and decomposition by solvents, especially polar organic solvents.

In accordance with the number of the units (C) in the copolymers of the invention it is possible to tailor the properties, such as peel strength and detachment resistance, printability, tensile strength and tear strength, or water-vapor permeability, for example.

The copolymers of the invention can be prepared by analogy with any desired processes which are already known to the skilled worker and which are used, for example, for the synthesis of (pre)polymers for polyurethanes.

The present invention further provides a process for preparing the inventive copolymers of the formula (II) by reacting

a) at least one compound of the formula

H-ND-Y—Si(OR¹)ₒR₂₋ₒ—(O—SiR_{q}R⁵_{2-q})ₙ—O—Si(OR¹)ₒR₂₋ₒ—Y-ND-H (VI),

b) at least one diisocyanate of the formula

OCN-Z(L)_{r′}—NCO (I)

where r′ is a number of at least 1 and/or reaction products thereof with phenols, ketoximes, malonic esters or nitrogen-containing heterocycles,
c) if desired diisocyanates of the formula

OCN-Z-NCO (VIII)

and/or blocked diisocyanates, such as the thermally unstable reaction products of isocyanates with, for example, phenols, ketoximes, malonic esters or nitrogen-containing heterocycles,
d) if desired a compound of the formula

H—NR⁴-G-NR⁴—H (IX),

e) if desired compounds of the formula

H-E-X-E-H (X),

f) if desired a catalyst, and
g) if desired at least one solvent, X, Y, Z, L, D, E, G, R, R¹, R⁴, R⁵, o and q being as defined above.

Examples of the inventively used compounds of the formula (VI) are α,ω-aminopropyldimethylsilyl-terminated polydimethylsiloxanes, α,ω-aminopropyl-dimethoxysilyl-terminated polydimethylsiloxanes, α,ω-aminomethyldimethylsilyl-terminated polydimethyl-siloxanes, and α,ω-aminomethyldimethoxysilyl-terminated polydimethylsiloxanes.

Preferred compounds of the formula (IX) which are used in the process of the invention are 3-(2-amino-ethyl)aminopropyltrimethoxysilane, 3-(2-amino-ethyl)aminopropyltriethoxysilane, 3-(2-amino-ethyl)aminopropylmethyldimethoxysilane, 3-(2-amino-ethyl)aminopropyldimethylmethoxysilane, 3-(2-amino-ethyl)aminopropylmethyldiethoxysilane, N,N′-bis(3-tri-methoxysilylpropyl)ethylenediamine, N,N′-bis(3-tri-ethoxysilylpropyl)ethylenediamine, N,N′-bis(3-dimethoxymethylsilylpropyl)ethylenediamine, and N,N′-bis(3-diethoxymethylsilylpropyl)ethylenediamine, N-trimethoxysilylmethylethylenediamine, N-triethoxysilylmethylethylenediamine, N-dimethoxymethylsilylmethylethylenediamine, N-diethoxymethylsilylmethylethylenediamine, and N-methoxydimethylsilylmethylethylenediamine.

Examples of the inventively used isocyanates of the formula (VIII) are hexylene diisocyanate, 4,4′-methylenedicyclohexylene diisocyanate, 4,4′-methylenediphenylene diisocyanate, 1,3-diazetidine-2,4-dione bis(4,4′-methylenedicyclohexyl)diisocyanate, 1,3-diazetidine-2,4-dione bis(4,4′-methylenediphenyl)-diisocyanate, and isophorone diisocyanate, preference being given to hexylene diisocyanate, 4,4′-methylene-dicyclohexylene diisocyanate, 4,4′-methylenediphenylene diisocyanate, and isophorone diisocyanate, and particular preference to hexylene diisocyanate, 4,4′-methylenedicyclohexylene diisocyanate, and isophorone diisocyanate.

The invention further provides diisocyanates of the general formula

(L)r′Z(NCO)2 (I),

where L can be alike or different and has a definition specified above for it,
Z has a definition specified above for it, and
r′ is a number of at least 1.

Examples of the inventive isocyanates of the formula (I) are N′—[H2C=C(CH3)C(═O)—O(CH2)2N(C(CH3)3)C(═O)NH—(CH2)6]-N″,N′″[(O═C═N—(CH2)6]2-cyclo[N(C═O)—N(C═O)—N(C═O)], N′—[H2C═C(CH3)C(═O)—O(CH2)2OC(═O)NH—(CH2)6]-N″,N′″[(O═C═N—(CH2)6]2-cyclo[N(C═O)—N(C═O)—N(C═O)], N′—[H2C=CH—C(═O)—O(CH2)2OC(═O)NH—(CH2)6]-N″,N′″[(O═C═N—(CH2)6]2-cyclo[N(C═O)—N(C═O)—N(C═O)], HC(C6H4N=C═O)2(C6H4NH—C(═O)—N(C(CH3)3)-(CH2)2-O—C(═O)—C(CH3)=CH2, HC(C6H4N═C═O)2(C6H4NH—C(═O)—O—(CH2)2-O—C(═O)—C(CH3)=CH2, and HC(C6H4N=C═O)2(C6H4NH—C(═O)—O —(CH₂)₂—O—C(═O)—CH═CH₂.

The invention additionally provides a process for preparing the diisocyanates of the formula (I), characterized in that Z(NCO)_{2+r′} (compound 1) is reacted with HE-Q-E-(C═O)—CR⁶═CR⁷₂(compound 2), optionally in the presence of a catalyst, with r′, Z, E, Q, R⁶, and R⁷ having one of the definitions described above.

Examples of the inventively employed compound 2 are 4-hydroxy-n-butyl acrylate, 4-hydroxy-n-butyl methacrylate, 3-hydroxy-n-butyl acrylate, 3-hydroxy-n-butyl methacrylate, 3-hydroxy-n-propyl acrylate, 3-hydroxy-n-propyl methacrylate, 2-hydroxy-n-propyl acrylate, 2-hydroxy-n-propyl methacrylate, hydroxypentyl acrylate and methacrylate, hydroxyhexyl acrylate and methacrylate, N-hydroxymethylacrylamide, N-hydroxymethylmethacrylamide, glyceryl dimethacrylate, 2-(tert-butylamino)ethyl methacrylate, hydroxyoctyl acrylate and methacrylate, the reaction product of 1 mol of hydroxyethyl acrylate and/or hydroxyethyl methacrylate with on average 2 mol of ∈-caprolactone and/or the reaction product of acrylic acid and/or methacrylic acid with the glycidyl ester of a carboxylic acid with a tertiary α carbon atom. These glycidyl esters of carboxylic acids have 11 to 13 carbon atoms and are available commercially, e.g. Cardura® from Shell.

Examples of the catalysts employed where appropriate are all of the catalysts known to date that promote the addition of the isocyanate groups of the compounds 1 onto the active groups of the compound 2, such as diorganotin compounds and bismuth compounds, for instance.

The compound 1 and compound 2 employed in the process of the invention are standard commercial products and/or can be prepared by methods that are known in chemistry.

Examples of the compounds of the formula (X) inventively used are compounds known from polyurethane chemistry, such as diols, such as ethylene glycol, polyethylene glycols, polypropylene glycols, polyester polyols, diamines such as, for example, ethylene-diamine, 5-amino-3-(aminomethyl)-1,3,3-trimethylcyclo-hexane, bis(4-amino-3-methylphenyl)methanes, isomer mixture of diaminodiethylmethylbenzene, bis(4-amino-3-chlorophenyl)methane, 2-methylpropyl 4-chloro-3,5-diaminobenzoate and amino-terminated polyethers (ATPE), for example.

The stoichiometry of the reactants for preparing the copolymers of the invention is preferably selected such that the molar ratio of the isocyanate groups from the compounds of the formula (VII) and (VIII) to the sum of the EH and NH groups, reactive with the isocyanate groups, from the compounds of the formulae (VI), (IX), and (X) is in the range from preferably 0.7 to 1.3, more preferably 0.95 to 1.05, in particular 1. With a ratio of the isocyanate groups to the reactive groups of greater than 1, i.e., an excess of isocyanate groups, inventive polymers of the formula (II) are produced with R′═NH—CO-Z(L)ᵣ-NCO and the resultant radicals R′ as defined above at the other end of the polymer chains. With a ratio less than 1, i.e., a deficit amount of isocyanate groups, inventive polymers of the formula (II) are produced with R″═H and the resultant radicals R′ as defined above at the other end of the polymer chains.

Examples of the catalysts used if desired are all catalysts known to date which promote the addition of the isocyanate groups of the compounds of the general formulae (VII) and (VIII) onto the active groups of the polymers in accordance with the formulae (VI), (IX) and (X), such as, for instance, diorganotin compounds and bismuth compounds.

With particular preference no catalysts are used in the process of the invention.

If catalysts are used in the process of the invention, the amounts involved are from preferably 0.0001 to 1 part by weight, more preferably 0.001 to 0.1 part by weight, based in each case on 100 parts by weight of the total mixture.

Examples of the solvents used if desired in the process of the invention are tetrahydrofuran, dimethyl-formamide, isopropanol, and methyl ethyl ketone.

With particular preference no solvents are used in the process of the invention.

If solvents are used in the process of the invention, the amounts involved are from preferably 10 to 200 parts by weight, more preferably 10 to 100 parts by weight, based in each case on 100 parts by weight of total mixture.

The reactants used in the process of the invention for preparing copolymers of the formula (II), with the exception of the diisocyanate of the formula (I), are standard commercial products and/or can be prepared by methods that are commonplace in chemistry.

The reaction of the invention may take place in solution or in bulk (without solvent), preference being given to a reaction in bulk.

If the reaction of the invention takes place in solution, temperatures are preferably from 0 to 100° C. and with particular preference from 20 to 80° C.

If the reaction of the invention takes place in bulk, temperatures above the softening point of the copolymer of the formula (II) prepared are preferred.

In the case of a discontinuous operation, the process of the invention is carried out preferably under the pressure of the surrounding atmosphere, i.e., at between 900 and 1100 hPa. In the case of continuous preparation, in a twin-screw extruder, for example, the process is operated at a pressure of preferably up to 15 MPa in some sections of the extruder and, for devolatilization, at pressures of preferably 0.1 to 1100 hPa.

The process of the invention is carried out preferably in the absence of moisture and actinic radiation, although it is also possible to operate in the presence of water and radiation.

The preparation of the inventive copolymers in accordance with the general formula (II) can take place with methods which are known to the skilled worker, such as by means of extruders, compounders, roll mills, dynamic mixers or static mixers, for example. The copolymers of the invention can be prepared continuously or batchwise. Preferably the preparation is carried out continuously.

The siloxane copolymers prepared in accordance with the invention can then be freed by any desired processes known to date from any reactants that may still be present and/or from any solvents and/or catalysts that may have been used, by means of distillation or extraction, for example.

The components used in the process of the invention may in each case be one kind of such a component or else a mixture of at least two kinds of a respective component.

The process of the invention has the advantage that it is simple to implement and that a large number of possible copolymers can be prepared with great variability.

The process of the invention has the advantage, moreover, that copolymers can be prepared in a well-defined way.

The copolymers of the invention, of the formula (II), can be prepared and processed using the typical processing methods for radiation-crosslinkable and, where appropriate, moisture-crosslinkable polymers and/or thermoplastic elastomers—for example, by means of extrusion, injection molding, blow molding, vacuum thermoforming. Processing in the form of a solution or emulsion or suspension is a further possibility.

Preferred applications of the inventive or inventively prepared copolymers of the formula (II) are uses as a constituent in adhesives and sealants, as a base material for thermoplastic elastomers such as cable sheathing, hoses, seals, and keyboard mats, for example, for membranes, such as selectively gas-permeable membranes, as additives in polymer blends, or for coating applications, for example, in antistick coatings, tissue-compatible coatings, flame-retardant coatings and as biocompatible materials. Further application possibilities are as sealants and adhesives, such as hotmelt adhesives, for example, adhesives for application as a solution, primers for improving the adhesion of sealants and adhesives to different substrates, additives for polymer processing, antifouling coatings, cosmetics, bodycare products, paint additives, an auxiliary in laundry detergents and in the treatment of textiles, for the modification of resins, or for bitumen modification.

The use of the inventive or inventively prepared copolymers is possible in numerous applications, such as, for example, in sealants, adhesives, as material for modifying fibers, as plastics additive, for example, as impact modifiers or flame retardants, as material for defoamer formulations, as a high-performance polymer (thermoplastic, thermoplastic elastomer, elastomer), as packaging material for electronic components, in insulation materials or shielding materials, in cable sheathing, in antifouling materials, as an additive for scouring, cleaning or polishing products, as an additive for bodycare compositions, as a coating material for wood, paper, and paperboard, as a mold release agent, as a biocompatible material in medical applications such as contact lenses, as a coating material for textile fibers or textile fabric, as a coating material for natural substances such as leather and furs, for example, as material for membranes, and as material for photoactive systems—for lithographic techniques, flexographic printing plates, optical data securement or optical data transmission, for example.

Preference extends to the use of the copolymers of the invention as a release coating for adhesive tapes and labels, fiber coating for textiles, for example, extrusion aid for thermoplastics processing, medical devices, such as catheters, infusion bags or infusion tubes, for example, hotmelt adhesives, PSA coatings, components for the automobile industry that can be overpainted and oversprayed, an additive for polymer modification, such as plasticizers or impact modifiers, for example, film for laminated safety glass, or joint sealant for the construction industry.

The copolymers of the invention can be employed wherever organopolysiloxane-polyurea copolymers have been employed to date.

The copolymers of the formula (II) prepared in accordance with the invention are especially suitable for use in crosslinkable compositions, such as photocrosslinkable compositions, for instance.

The present invention further provides crosslinkable compositions comprising inventive or inventively prepared copolymers of the formula (II).

The crosslinkable compositions of the invention are preferably compositions crosslinkable by actinic radiation.

Particular preference is given to crosslinkable compositions comprising

(i) copolymer of the formula (II), if desired
(ii) crosslinker, if desired
(iii) a photopolymerization initiator, if desired
(iv) filler, if desired
(v) adhesion promoter, if desired
(vi) further substances selected from the group containing plasticizers, stabilizers, antioxidants, flame retardants, light stabilizers, and pigments, and if desired
(vii) crosslinkable polymers different to (i).

These crosslinkable compositions of the invention are preferably one-component compositions. To prepare these one-component compositions it is possible for the constituents respectively used to be mixed with one another in any desired manner known to date. This mixing takes place preferably at room temperature or at a temperature which comes about when the constituents are combined at room temperature, without additional heating or cooling, and at the pressure of the surrounding atmosphere, in other words about 900 to 1100 hPa. Alternatively, if desired, this mixing can take place at higher or lower pressures—at lower pressures, for example, in order to avoid gas inclusions.

The preparation of the compositions of the invention and their storage take place preferably under substantially radiation-free and, if desired, anhydrous conditions, in order to prevent premature reaction of the compositions.

Crosslinkers (ii) employed if desired can be any crosslinkers which have also been employed to date in radiation-crosslinkable compositions, such as crosslinkers containing co-crosslinkable, radiation-curable, unsaturated C═C bonds. Crosslinkers (ii) are preferably acrylates.

Examples of crosslinkers (ii), which are employed if desired, are monofunctional oligo(ethers) and monomeric acrylates and methacrylates, such as 2-(2-ethoxy-ethoxy)ethyl acrylate, 2-phenoxyethyl acrylate, caprolactone acrylate, cyclic trimethylolpropane formal acrylate, ethoxylated nonylphenol acrylate, isobornyl acrylate, isodecyl acrylate, lauryl acrylate, octyldecyl acrylate, stearyl acrylate, tetrahydro-furfuryl acrylate, tridecyl acrylate, 2-phenoxyethyl methacrylate, ethoxylated hydroxyethyl methacrylate, isobornyl methacrylate, lauryl methacrylate, methoxypolyethylene glycol(350) monomethacrylate, methoxypolyethylene glycol(550)monomethacrylate, polypropylene glycol monomethacrylate, stearyl methacrylate, tetrahydrofurfuryl methacrylate; difunctional oligo(ethers) and monomeric acrylates and methacrylates, such as 1,6-hexanediol diacrylate, alkoxylated diacrylates, alkoxylated hexanediol diacrylates, diethylene glycol diacrylate, dipropylene glycol diacrylate, ester diol diacrylate, ethoxylated bisphenol A diacrylates, polyethylene glycol(200) diacrylate, polyethylene glycol(400) diacrylate, polyethylene glycol(600) diacrylate, propoxylated neopentyl glycol diacrylate, tetraethylene glycol diacrylate, tricyclodecanedimethanol diacrylate, triethylene glycol diacrylate, tripropylene glycol diacrylate, 1,3-butylene glycol dimethacrylate, 1,4-butanediol dimethacrylate, 1,6-hexandiol dimethacrylate, diethylene glycol dimethacrylate, ethoxylated bisphenol A dimethacrylates, ethylene glycol dimethacrylate, polyethylene glycol(200) dimethacrylate, polyethylene glycol(400) dimethacrylate, polyethylene glycol(600) dimethacrylate, tetraethylene glycol dimethacrylate, triethylene glycol dimethacrylate; trifunctional and higher polyfunctional oligo(ethers) and monomeric acrylates and methacrylates, such as dipentaerythritol pentaacrylate, ditrimethylolpropane tetraacrylate, ethoxylated trimethylolpropane triacrylates, pentaerythritol tetraacrylate, pentaerythritol triacrylate, propoxylated glycerol triacrylates, propoxylated trimethylolpropane triacrylate, trimethylolpropane triacrylate, tris(2-hydroxyethyl)isocyanurate triacrylate, trimethylol-propane trimethacrylate;

epoxy acrylates, such as bisphenol A epoxyacrylate, epoxidized soybean oil acrylate, epoxy novolac acrylate oligomer, fatty acid-modified bisphenol A epoxy-acrylate;
aliphatic and aromatic urethane acrylates and polyester acrylates;
silanes containing SiC-bonded vinyl, allyl, acryloyloxy, methacryloyloxy groups and also their partial hydrolyzates and cohydrolyzates;
styrene, isoprene, butadiene and vinyl acetate.

If the crosslinkable compositions of the invention comprise crosslinkers (ii), the amounts involved are from preferably 0.05 to 70 parts by weight, more preferably 0.2 to 30 parts by weight, based in each case on 100 parts by weight of crosslinkable composition.

As photopolymerization initiators (iii) employed it is possible to use all of the initiators known to the skilled worker, or mixtures thereof.

Examples of initiators (iii) used are benzyl dimethyl ketal, 2-hydroxy-2-methylphenylpropan-1-one, 1-hydroxy-cyclohexyl phenyl ketone, isopropylthioxanthone, bisacylphosphine oxide, 1-[4-(2-hydroxyethoxy)phenyl]-2-hydroxy-2-methylpropan-1-one, benzoin n-butyl ether, polymeric hydroxyketones, such as oligo(2-hydroxy-2-methyl-1,4-(1-methylvinyl)phenylpropanone), acenaphthylquinone, α-aminoacetophenone, benzanthraquinone, benzoin methyl ether, benzoin isopropyl ether, benzoin isobutyl ether, benzophenone, benzyl dimethyl acetal, benzyl 1-methyl-1-ethyl acetal, 2,2-diethoxy-2-phenyl-acetophenone, 2,2-diethoxyacetophenone, 2-dimethoxy-benzoyldiphenylphosphine oxide, 2,2-dimethoxy-2-phenyl-acetophenone, i.e., Irgacure® 651 (Ciba-Geigy), 4,4-bis(dimethylamino)benzophenone, 2-ethylanthraquinone, ethyl 2,4,6-trimethylbenzoylphenylphosphinate, hydroxy-acetophenone, 2-hydroxy-2-methylpropiophenone, 2-hydro-oxy-2-methyl-4′-isopropylisopropiophenone, 1-hydroxy-ycyclohexyl phenyl ketone, 4′-morpholinodeoxybenzoin, 4-morpholinobenzophenone, α-phenylbutyrophenone, 2,4,6-trimethylbenzoyldiphenylphosphine oxide, and 4,4′-bis(dimethylamino)benzophenone.

A photopolymerization initiator can also be used in combination with coinitiators, such as ethyl-anthraquinone with 4,4′-bis(dimethylamino)benzophenone, benzoin methyl ether with triphenylphosphine, benzyl dimethyl ketal with benzophenone, diacylphosphine oxides with tertiary amines, or acyldiarylphosphine oxides with benzyl dimethyl acetal.

If the crosslinkable compositions of the invention comprise photopolymerization initiator (iii), the amounts involved are from preferably 0.01 to 5 parts by weight, more preferably 0.05 to 3 parts by weight, based in each case on 100 parts by weight of crosslinkable composition.

Fillers (iv) employed if desired can be any fillers which have also been employed to date in crosslinkable compositions. Examples of fillers are reinforcing fillers, which are fillers having a BET surface area of at least 30 m²/g, such as carbon blacks, fumed silica, precipitated silica, and silicon-aluminum mixed oxides, it being possible for said fillers to have been hydrophobicized, and also nonreinforcing fillers, which are fillers having a BET surface area of less than 30 m²/g, such as powders of quartz, cristobalite, diatomaceous earth, calcium silicate, zirconium silicate, montmorillonites, such as bentonites, zeolites, including the molecular sieves, such as sodium aluminum silicate, metal oxides, such as aluminum oxide or zinc oxide and/or their mixed oxides, metal hydroxides, such as aluminum hydroxide, barium sulfate, calcium carbonate, gypsum, silicon nitride, silicon carbide, boron nitride, glass powder, carbon powder, and polymer powders, and hollow glass and plastic beads.

Filler (iv) preferably comprises fumed silicas, a BET surface area of at least 30 m²/g being particularly preferred.

If the compositions of the invention comprise fillers (iv), the amounts involved are from preferably 1 to 50 parts by weight, preferably 2 to 30 parts by weight, based in each case on 100 parts by weight of crosslinkable composition.

As adhesion promoters (v), which can be employed if desired, it is possible to employ any adhesion promoters which have also been employed to date in radiation-crosslinkable compositions. Examples of adhesion promoters (v) are silanes containing SiC-bonded vinyl, acryloyloxy, methacryloyloxy groups and also their partial hydrolyzates and cohydrolyzates, and acrylates such as 2-(2-ethoxyethoxy)ethyl acrylate, 2-phenoxyethyl acrylate, cyclic trimethylolpropane formal acrylate, 1,6-hexanediol diacrylate, pentaerythritol tetraacrylate, tetrahydrofurfuryl meth-acrylate, methoxy-polyethylene glycol(550) monomethacrylate, and stearyl methacrylate.

If the compositions of the invention comprise adhesion promoters (v), the amounts involved are from preferably 0.01 to 5 parts by weight, more preferably 0.5 to 4 parts by weight, based in each case on 100 parts by weight of crosslinkable composition.

Examples of further substances (vi) are plasticizers, such as trimethylsilyl-terminated polydimethylsiloxanes and hydrocarbons having about 16 to 30 carbon atoms, stabilizers, such as 2-ethylhexyl phosphate, octylphosphonic acid, polyethers, antioxidants, flame retardants, such as phosphoric esters, light stabilizers, and pigments, such as titanium dioxide and iron oxides.

The further substances (vi), employed if desired, are preferably plasticizers, such as trimethylsilyl-terminated polydimethylsiloxanes and hydrocarbons having about 16 to 30 carbon atoms, stabilizers, such as 2-ethylhexyl phosphate, octylphosphonic acid, polyethers, flame retardants, such as phosphoric esters, and pigments, such as titanium dioxide and iron oxides, particular preference being given to stabilizers and pigments.

If constituent (vi) is employed, the amounts involved are from preferably 0.01 to 30 parts by weight, more preferably from 0.05 to 25 parts by weight, based in each case on 100 parts by weight of crosslinkable composition.

The crosslinkable compositions of the invention may if desired comprise crosslinkable polymers (vii), such as organopolysiloxanes having reactive groups, and aliphatic and aromatic urethane acrylates and polyester acrylates. Examples of such crosslinkable siloxanes are α,ω-diacryloyloxymethylpolydimethylsiloxanes, α,ω-divinylpolydimethylsiloxanes, poly(dimethyl-co-methyl-vinyl)siloxanes and α,ω-methacryloyloxypropyl-terminated polydimethylsiloxanes.

Component (vii) employed if desired in the crosslinkable compositions of the invention preferably comprises polydiorganosiloxanes having at least one radiation-crosslinkable group at the chain ends, more preferably polydimethylsiloxanes having at least one acrylic group at the chain ends and poly(dimethyl-co-methylvinyl)siloxanes, particularly α,ω-diacryloyloxy-methylpolydimethylsiloxanes, α,ω-methacryloyloxy-propyl-terminated polydimethylsiloxanes, and poly-(dimethyl-co-methylvinyl)siloxanes having a viscosity of 100 to 500 000 mPas.

The crosslinkable compositions of the invention preferably comprise component (vii). This constituent is used preferably for adjusting the processing properties, such as the viscosity, for example.

If component (vii) is used, the amounts involved are from preferably 1 to 50 parts by weight, more preferably 2 to 25 parts by weight, based in each case on 100 parts by weight of crosslinkable composition.

The individual constituents of the crosslinkable compositions of the invention may in each case comprise one kind of such a constituent or else a mixture of at least two different kinds of such constituents.

In particular the compositions of the invention contain no further constituents apart from component (i), if desired (ii), (iii), (iv), (v), (vi), and (vii).

The preparation of the crosslinkable compositions of the invention takes place with methods which are known to the skilled worker, such as by means of extruders, compounders, roll mills, dynamic mixers or static mixers, for example. The compositions of the invention can be prepared continuously or batchwise. Preferably the preparation takes place continuously.

Vulcanizates of the compositions of the invention can be obtained by irradiation. Radiation sources, such as UV lamps, lasers, and sunlight, are known to the person skilled in the art. The irradiation wavelengths and irradiation times are tailored to the photopolymerization initiators used and to the compounds to be polymerized. The compositions of the invention are crosslinked preferably at room temperature. If desired, crosslinking may also take place at temperatures higher or lower than room temperature, such as at −50 to 15° C. or at 30 to 150° C., for example. Preferably the crosslinking is carried out at a pressure of 100 to 1100 hPa, in particular at the pressure of the surrounding atmosphere, in other words about 900 to 1100 hPa.

The present invention further provides moldings produced by crosslinking the compositions of the invention.

In comparison to non-crosslinked thermoplastic siloxane-urea copolymers of the prior art, the vulcanizates of the copolymers of the invention have, after radiation crosslinking, a lower dependence of the mechanical properties on the temperature. Through crosslinking, the vulcanizates of the copolymers of the invention lose their plasticity when the temperature is increased, and so are no longer able to flow and are therefore more dimensionally stable. All in all, therefore, the vulcanizates of the invention have better mechanical properties over a wider temperature range, and so can be used in more diverse fields of use.

The crosslinkable compositions of the invention are used preferably as an adhesive, hotmelt adhesive, PSA (pressure-sensitive adhesive), sealant, coating for substrates including paper, textile, fibers or silicatic surfaces, for example, impregnating agent, paint, constituent in composite materials, additive for polymers, molding, and component for medical use, and also for use in automobile construction or laminated glass.

The compositions of the invention have the advantage that they possess all of the abovementioned advantages of the copolymers of the invention that are used.

The compositions of the invention have the advantage that they exhibit very good mechanical properties.

Further advantages of the compositions of the invention are the high thermal and oxidative stability, good stabilities toward swelling and decomposition by polar organic solvents.

The compositions of the invention have the advantage that the properties, such as peel strength and detachment resistance, printability, tensile strength and tear strength, or water-vapor permeability, for example, can be tailored.

The moldings of the invention have the advantage of possessing a relatively low dependence of the mechanical properties on the temperature, in particular at relatively high temperatures.

The moldings of the invention have the advantage, moreover, of possessing a very good adhesion to substrates.

In the examples described below, all viscosity data relate to a temperature of 25° C. Unless indicated otherwise, the examples below are carried out at the pressure of the surrounding atmosphere, in other words at about 1000 hPa, and at room temperature, in other words at about 23° C., or at a temperature which comes about when the reactants are combined at room temperature without additional heating or cooling, and also at a relative atmospheric humidity of about 50%. Moreover, all parts and percentages data, unless otherwise indicated, relate to the weight.

The Shore A hardness is determined in accordance with DIN (Deutsche Industrie Norm [German Industry Standard]) 53505 (August 2000 edition).

Tensile strength (TS), breaking elongation (BE) and modulus (stress at 100% elongation) were determined in accordance with DIN 53504 (May 1994 edition) on specimens of shape S2.

In the examples below, the irradiation took place using a xenon lamp in an instrument of the type “Heraeus Suntest CPS” (550 W/m²) from Atlas Material Testing Technology GmbH (63589 Linsengericht, Germany) with a filter >290 nm (called “UV lamp” below).

### EXAMPLE 1

18.3 g of the trimer of hexamethylene diisocyanate (available commercially under the brand name Desmodur® N3600 from Bayer AG, Germany) are stirred with 3.9 g of hydroxyethyl acrylate and 20 mg of a bismuth catalyst (available commercially under the name Borchi® Kat VP 0244 from Borchers GmbH, 40764 Langenfeld, Germany) in the absence of light at 40° C. for 4 hours. The system is stabilized by addition of 1000 ppm of 2,6-di-tert-butyl-4-methylphenol. The identity of the product as a monoacrylate and diisocyanate is confirmed by means of ¹³C NMR spectroscopy.

### EXAMPLE 2

18.3 g of the trimer of hexamethylene diisocyanate (available commercially under the brand name Desmodur® N3600 from Bayer AG, Germany) are stirred with 4.5 g of hydroxyethyl methacrylate and 20 mg of a bismuth catalyst (available commercially under the name Borchi® Kat VP 0244 from Borchers GmbH, 40764 Langenfeld, Germany) in the absence of light at 40° C. for 4 hours. The system is stabilized by addition of 1000 ppm of 2,6-di-tert-butyl-4-methylphenol. The identity of the product as a monoacrylate and diisocyanate is confirmed by means of ¹³C NMR spectroscopy.

### EXAMPLE 3

18.3 g of the trimer of hexamethylene diisocyanate (available commercially under the brand name Desmodur® N3600 from Bayer AG, Germany) are stirred with 6.4 g of 2-(tert-butylamino)ethyl methacrylate in the absence of light at 40° C. for 4 hours. The system is stabilized by addition of 1000 ppm of 2,6-di-tert-butyl-4-methylphenol. The identity of the product as a monoacrylate and diisocyanate is confirmed by means of ¹³C NMR spectroscopy.

### EXAMPLE 4

22.2 g of the product from example 1 are dissolved in 50 ml of tetrahydrofuran (THF) and the solution is added to 90 g of a solution, in 200 ml of THF, of polydimethylsiloxane terminated at both ends by 3-aminopropyl, this solution having a viscosity of 50 mPas. The solution is poured into a PTFE mold with a depth of approximately 2 mm and the solvent is evaporated in the absence of light. This procedure is repeated until sheets 2 mm thick are obtained, from which test specimens are punched. Mechanical data for S2 rods are given in table 1.

### EXAMPLE 5

66 g of the product produced in example 4 are dissolved in 400 g of tetrahydrofuran (THF). 14 g of a polydimethylsiloxane terminated at both ends by trimethylsilyl and having a viscosity of 10 mPas, 9.3 g of tetrahydrofurfuryl acrylate, and 1.12 g of ω,ω-dimethoxy-ω-phenylacetophenone (benzil dimethyl ketal) are mixed in. The solution is poured into a PTFE mold with a depth of approximately 2 mm and the solvent is evaporated in the absence of light. This procedure is repeated until sheets 2 mm thick are obtained, from which test specimens are punched. Mechanical data for S2 rods are given in table 1.

One of the sheets produced is irradiated for 1 minute while a second sheet is irradiated for 5 minutes with the UV lamp. Mechanical data for S2 rods are given in table 1.

TABLE 1

| | | | | Stress value at |
|---|---|---|---|---|
| | Hardness | TS | BE | 100% elongation |
| | [Shore A] | [MPa] | [%] | [MPa] |
| Example 4 | 42 | 1.45 | 76 | — |
| Example 5 | 21 | 0.71 | 151 | 0.56 |
| Example 5, | 38 | 0.84 | 34 | — |
| 1 min | | | | |
| irradiation | | | | |
| Example 5, | 43 | 0.78 | 40 | — |
| 5 mins | | | | |
| irradiation | | | | |

### EXAMPLE 6

8.37 g of the product from example 2 and 1.37 g of isophorone diisocyanate are dissolved in 30 ml of tetrahydrofuran (THF) and this solution is added to 40 g of a solution, in 200 ml of THF, of polydimethyl-siloxane terminated at both ends by 3-aminopropyl, this solution having a viscosity of 50 mPas. Mixed into the resulting solution are 12.4 g of a polydimethylsiloxane terminated at both ends by trimethylsilyl and having a viscosity of 10 mPas, 8.28 g of tetrahydrofurfuryl acrylate, and 0.99 g of ω,ω-dimethoxy-ω-phenyl-acetophenone (benzil dimethyl ketal). The solution is poured into a PTFE mold with a depth of approximately 2 mm and the solvent is evaporated in the absence of light. This procedure is repeated until sheets 2 mm thick are obtained, from which test specimens are punched. Mechanical data for S2 rods are given in table 2.

One of the sheets produced is irradiated for 1 minute while a second sheet is irradiated for 5 minutes with the UV lamp. Mechanical data for S2 rods are given in table 2.

TABLE 2

| | | | | Stress value at |
|---|---|---|---|---|
| | Hardness | TS | BE | 100% elongation |
| | [Shore A] | [MPa] | [%] | [MPa] |
| Example 6 | 5 | 0.43 | 733 | 0.17 |
| Example 6, | 46 | 0.97 | 59 | — |
| 1 min | | | | |
| irradiation | | | | |
| Example 6, | 48 | 0.80 | 46 | — |
| 5 mins | | | | |
| irradiation | | | | |

### EXAMPLE 7

24.7 g of the product from example 3 are dissolved in 50 ml of tetrahydrofuran (THF) and this solution is added to 90 g of a solution, in 200 ml of THF, of polydimethylsiloxane terminated at both ends by 3-aminopropyl, this solution having a viscosity of 50 mPas. Mixed into the solution are 13.1 g of a polydimethylsiloxane terminated at both ends by trimethylsilyl and having a viscosity of 10 mPas, 8.7 g of tetrahydrofurfuryl acrylate, and 1.05 g of ω,ω-dimethoxy-ω-phenylacetophenone (benzil dimethyl ketal). The solution is poured into a PTFE mold with a depth of approximately 2 mm and the solvent is evaporated in the absence of light. This procedure is repeated until sheets 2 mm thick are obtained, from which test specimens are punched. Mechanical data for S2 rods are given in table 3.

One of the sheets produced is irradiated for 1 minute while a second sheet is irradiated for 5 minutes with the UV lamp. Mechanical data for S2 rods are given in table 3.

TABLE 3

| | | | | Stress value at |
|---|---|---|---|---|
| | Hardness | TS | BE | 100% elongation |
| | [Shore A] | [MPa] | [%] | [MPa] |
| Example 7 | 25 | 0.76 | 136 | 0.69 |
| Example 7, | 46 | 1.01 | 43 | — |
| 1 min | | | | |
| irradiation | | | | |
| Example 7, | 48 | 1.05 | 36 | — |
| 5 mins | | | | |
| irradiation | | | | |

1-8. (canceled)

9. A siloxane/urea copolymer of the formula
R′-[(A)ₐ(B)_{b}(C)_{c}]-R″ (II),
in which
unit(s) (A) are alike or different and are unit(s) of the formula (III)
[CO—NH-Z(L)ᵣ-NH—CO-ND-Y—Si(OR¹)ₒR₂₋ₒ-(O—SiR_{q}R5_{2-q})ₙ—O—Si(OR¹)ₒR₂₋ₒ—Y-ND]-,
unit(s) (B) are alike or different and are unit(s) of the formula
—[CO—NH-Z(L)ᵣ-NH—CO—NR⁴-G-NR⁴]— (IV)
and
unit(s) (C) are alike or different and are unit(s) of the formula
—[CO—NH-Z(L)ᵣ-NH—CO-E-X-E]- (V)
where
X each independently is a C₁₋₇₀₀ alkylene radical which is unsubstituted or substituted by fluorine, chlorine, C₁₋₆ alkyl or C₁₋₆ alkyl ester and in which non-adjacent methylene units are optionally replaced by groups —O—, —COO—, —OCO— or —OCOO—, or is an unsubstituted or substituted C₆₋₂₂ arylene radical,
Y each independently is a divalent C₁₋₃₀ hydrocarbon radical in which non-adjacent methylene units are optionally replaced by group(s) —O—, or Y is the radical —(CH₂)₃—NH—SiR₂—(CH₂)₃—NH—,
Z each independently is an (r+2)-valent, unsubstituted or substituted C₁₋₆₀ hydrocarbon radical optionally interrupted by one or more heteroatoms,
L each independently is a radical —NH—(C═O)-E-Q-E-(C═O)—CR⁶═CR⁷₂,
Q each independently is a divalent, unsubstituted or substituted C₁₋₃₀ hydrocarbon radical optionally interrupted by one or more heteroatoms,
R⁶ each independently is a hydrogen atom or a monovalent C₁₋₂₀ hydrocarbon radical which is unsubstituted or substituted by fluorine or chlorine,
R⁷ each independently is a hydrogen atom or a monovalent C₁₋₂₀ hydrocarbon radical which is unsubstituted or substituted by fluorine or chlorine,
r each independently is 0 or an integer of at least 1,
D each independently is a hydrogen atom or a monovalent, unsubstituted or substituted hydrocarbon radical,
E each independently is an oxygen atom or an amino group —ND-,
R each independently is a monovalent C₁₋₂₀ hydrocarbon radical and is optionally substituted by fluorine or chlorine,
R⁵ each independently is a monovalent, C═C-unsaturated C₂₋₂₀ hydrocarbon radical optionally substituted by fluorine, chlorine or oxygen, and is optionally interrupted by oxygen,
q is 0, 1 or 2
R¹ each independently is a hydrogen atom or a monovalent C₁₋₂₀ hydrocarbon radical optionally substituted by fluorine, chlorine or organyloxy groups, or is —(C═O)—R or —N═CR₂,
R⁴ each independently is a radical of the formula -Z′-SiRₚ(OR¹)₃₋ₚ wherein p is 0, 1 or 2, or is a hydrogen atom or a monovalent, unsubstituted or substituted hydrocarbon radical,
Z′ each independently is a divalent, unsubstituted or substituted C₁₋₃₀ hydrocarbon radical,
G each independently is a divalent, unsubstituted or substituted C₁₋₆₀ hydrocarbon radical, optionally interrupted by one or more heteroatoms,
R″ is a hydrogen atom or a radical —CO—NH-Z(L)ᵣ-NCO,
R′ if R″ is a hydrogen atom, is a radical HND-Y—Si(OR¹)ₒR₂₋ₒ—(O—SiR₂)ₙ—O—Si(OR¹)ₒR₂₋ₒ—Y-ND-, HNR⁴-G-NR⁴—, or HE-X-E-, and if R″ is a radical —CO—NH-Z(L)ᵣ-NCO, R′ is a radical OCN-Z(L)ᵣ-NH—CO-ND-Y—Si(OR¹)ₒR₂₋ₒ—(O—SiR₂)ₙ—O—Si(OR¹)ₒR₂₋ₒ—Y-ND-, OCN-Z(L)ᵣ-NH—CO—NR⁴-G-NR⁴—, or OCN-Z(L)ᵣ-NH—CO-E-X-E-,
n each independently is an integer from 1 to 4000,
o each independently is 0, 1 or 2,
a is an integer of at least 1,
b is 0 or an integer of at least 1,
c is 0 or an integer of at least 1,
with the proviso that in the molecule there is at least one radical L, and wherein the individual blocks (A), (B) and (C) are distributed in the polymer in any order.

10. A process for preparing a copolymer of claim 1, comprising reacting
a) at least one compound of the formula
H-ND-Y—Si(OR¹)ₒR₂₋ₒ—(O—SiR_{q}R⁵_{2-q})ₙ—O—Si(OR¹)ₒR₂₋ₒ—Y-ND-H (VI),
b) at least one diisocyanate of the formula
OCN-Z(L)_{r′}—NCO (I)
where r′ is a number of at least 1, or reaction products thereof with phenols, ketoximes, malonic esters or nitrogen-containing heterocycles, or mixtures thereof,
c) optionally, one or more diisocyanates of the formula
OCN-Z-NCO (VIII)
or blocked diisocyanates or mixtures thereof,
d) optionally, one or more compounds of the formula
H—NR⁴-G-NR⁴—H (IX),
e) optionally, one or more compounds of the formula
H-E-X-E-H (X),
f) optionally, a catalyst,
optionally in the present of a solvent.

11. The process of claim 10, wherein at least one blocked isocyanate selected from the group consisting of thermally unstable reaction products of isocyanates with phenols, ketoximes, malonic esters and nitrogen-containing heterocycles is employed.

12. A diisocyanate suitable for use in the process of claim 10, of the formula
(L)_{r′}Z(NCO)₂ (I),
where L may be the same or different, and has a definition
r′ is a number of at least 1.

13. A process for preparing a diisocyanate of claim 12, comprising reacting Z(NCO)_{2+r′} with HE-Q-E-(C═O)—CR⁶═CR⁷₂, optionally in the presence of a catalyst.

14. A crosslinkable composition comprising at least one copolymer of claim 9.

15. A crosslinkable composition comprising at least one copolymer prepared by the process of claim 10.

16. The crosslinkable composition of claim 14, which is crosslinkable by actinic radiation.

17. The crosslinkable composition of claim 15, which is crosslinkable by actinic radiation.

18. The crosslinkable composition of claim 14, comprising at least one copolymer of the formula (II), a crosslinker, and a photopolymerization initiator.

19. The crosslinkable composition of claim 15, comprising at least one copolymer of the formula (II), a crosslinker, and a photopolymerization initiator.

20. A molding produced by crosslinking a composition of claim 14.

21. A molding produced by crosslinking a composition of claim 18.
